SEQUENCE LISTING (13 sequences)

SEQ ID NO: 1 - DNA, 858 bp, H. sapiens
  CDS (1)..(858)
atggccggcctcggccacccctccgccttcggccgggccacccacgccgtggtgcgggcgctacccgagtcgctctgccagcacgcgctgagaagcgccaagggcgaggaggtggacgtcgcccgcgcggaacggcagcaccagctctacgtgggcgtgctgggcagcaagctggggctgcaggtggtggagctgccggccgacgagagccttccggactgcgtcttcgtggaggacgtggccgtggtgtgcgaggagacggccctcatcacccgacccggggcgccgagccggaggaaggaggttgacatgatgaaagaagcattagaaaaacttcagctcaatatagtagagatgaaagatgaaaatgcaactttagatggcggagatgttttattcacaggcagagaattttttgtgggcctttccaaaaggacaaatcaacgaggtgctgaaatcttggctgatacttttaaggactatgcagtctccacagtgccagtggcagatgggttgcatttgaagagtttctgcagcatggctgggcctaacctgatcgcaattgggtctagtgaatctgcacagaaggcccttaagatcatgcaacagatgagtgaccaccgctacgacaaactcactgtgcctgatgacatagcagcaaactgtatatatctaaatatccccaacaaagggcacgtcttgctgcaccgaaccccggaagagtatccagaaagtgcaaaggtttatgagaaactgaaggaccatatgctgatccccgtgagcatgtctgaactggaaaaggtggatgggctgctcacctgctgctcagttttaattaacaagaaggtagactcctga

SEQ ID NO: 2 - protein, 285 aa, H. sapiens
MAGLGHPSAFGRATHAVVRALPESLCQHALRSAKGEEVDVARAERQHQLYVGVLGSKLGLQVVELPADESLPDCVFVEDVAVVCEETALITRPGAPSRRKEVDMMKEALEKLQLNIVEMKDENATLDGGDVLFTGREFFVGLSKRTNQRGAEILADTFKDYAVSTVPVADGLHLKSFCSMAGPNLIAIGSSESAQKALKIMQQMSDHRYDKLTVPDDIAANCIYLNIPNKGHVLLHRTPEEYPESAKVYEKLKDHMLIPVSMSELEKVDGLLTCCSVLINKKVDS

SEQ ID NO: 3 - DNA, 858 bp, H. sapiens
  CDS (1)..(858)
atggggacgccgggggaggggctgggccgctgctcccatgccctgatccggggagtcccagagagcctggcgtcgggggaaggtgcgggggctggccttcccgctctggatctggccaaagctcaaagggagcacggggtgctgggaggtaaactgaggcaacgactggggctacagctgctagaactgccacctgaggagtcattgccgctgggaccgctgcttggcgacacggccgtgatccaaggggacacggccctaatcacgcggccctggagccccgctcgtaggccagaggtcgatggagtccgcaaagccctgcaagacctggggctccgaattgtggaaataggagacgagaacgcgacgctggatggcactgacgttctcttcaccggccgggagtttttcgtaggcctctccaaatggaccaatcaccgaggagctgagatcgtggcggacacgttccgggacttcgccgtctccactgtgccagtctcgggtccctcccacctgcgcggtctctgcggcatggggggacctcgcactgttgtggcaggcagcagcgacgctgcccaaaaggctgtccgggcaatggcagtgctgacagatcacccatatgcctccctgaccctcccagatgacgcagctgctgactgtctctttcttcgtcctgggttgcctggtgtgccccctttcctcctgcaccgtggaggtggggatctgcccaacagccaggaggcactgcagaagctctctgatgtcaccctggtacctgtgtcctgctcagaactggagaaagctggcgccgggctcagctccctctgcttggtgctcagcacacgcccccacagctga

SEQ ID NO: 4 - protein, 285 aa, H. sapiens
MGTPGEGLGRCSHALIRGVPESLASGEGAGAGLPALDLAKAQREHGVLGGKLRQRLGLQLLELPPEESLPLGPLLGDTAVIQGDTALITRPWSPARRPEVDGVRKALQDLGLRIVEIGDENATLDGTDVLFTGREFFVGLSKWTNHRGAEIVADTFRDFAVSTVPVSGPSHLRGLCGMGGPRTVVAGSSDAAQKAVRAMAVLTDHPYASLTLPDDAAADCLFLRPGLPGVPPFLLHRGGGDLPNSQEALQKLSDVTLVPVSCSELEKAGAGLSSLCLVLSTRPHS

SEQ ID NO: 5 - DNA, 777 bp, S. coelicolor
  CDS (1)..(777)
gtgcccagcaagaaggccctggtccgccgccccagccccaggctcgccgaaggactggtgacacacgtcgagcgggagcaggtcgatcacggcctggccctcgaacagtgggacgcctacgtcgaggccctcggagcacacggctgggagactctggaggtggacccggccgagtactgtccggactcggtcttcgtcgaggacgccgtcgtcgtgttccgcaacgtcgcgctgatcacgcggcccggcgccgagtcgcggcgcgcggagacggccggcgtcgaggaggccgtggcccggctcggctgctcggtgaactgggtgtgggagccgggcaccctggacggcggcgacgtcctgaagatcggcgacacgatctacgtgggacgcggcggccggaccaacgcggccggtgtccagcagttgcgggcggcgttcgagccgctgggcgcccgggtcgtcgccgtgcccgtgagcaaggtgctgcatctgaagtcggcggtcaccgcgctgccggacgggacggtgatcgggcacatcccgctgacggacgtgccctcgctgttcccccgtttcctgccggtgccggaggagtcgggggcgcacgtggtgctgctcggcgggagcaggctgctgatggcggcgagcgcgcccaagacggcggagctgctcgccgatctcggtcacgagccggtgctcgtcgacatcggggagttcgagaagctggagggctgtgtgacgtgcctctcggtcaggctgcgcgagctgtacgactga

SEQ ID NO: 6 - protein, 258 aa, S. coelicolor
VPSKKALVRRPSPRLAEGLVTHVEREQVDHGLALEQWDAYVEALGAHGWETLEVDPAEYCPDSVFVEDAVVVFRNVALITRPGAESRRAETAGVEEAVARLGCSVNWVWEPGTLDGGDVLKIGDTIYVGRGGRTNAAGVQQLRAAFEPLGARVVAVPVSKVLHLKSAVTALPDGTVIGHIPLTDVPSLFPRFLPVPEESGAHVVLLGGSRLLMAASAPKTAELLADLGHEPVLVDIGEFEKLEGCVTCLSVRLRELYD

SEQ ID NO: 7 - DNA, 765 bp, P. aeruginosa
  CDS (1)..(765)
atgttcaagcacatcatcgctcgcacgcccgcccgcagcctggtcgacggcctgacctccagccacctcggcaagccggactacgccaaggccctggagcagcacaacgcctacatccgcgccttgcagacctgcgacgtggacatcaccctgctgccgccggacgaacgcttccccgactcggtgttcgtcgaggacccggtgctctgcacctcgcgctgcgccatcatcacccgccccggcgccgaatcgcggcgcggcgagaccgagatcatcgaggaaaccgtgcagcgcttctatccgggcaaggtcgagcgcatcgaggcacccggcacggtggaagccggcgacatcatgatggtcggcgaccacttctacatcggcgaatcggcccgcaccaacgccgagggcgcccggcagatgatcgcgatcctggagaaacatggcctcagcggctcggtggtgcgcctggaaaaggtcctgcacctgaagaccgggctcgcctacctggaacacaacaacctgctggccgccggcgagttcgtcagcaagccggagttccaggacttcaacatcatcgagatccccgaagaggagtcctacgccgccaactgcatctgggtcaacgaaagggtgatcatgcccgccggctatccccggacccgcgagaagatcgcccgcctcggctaccgggtgatcgaggtggacacctccgaatatcgcaagatcgacggcggcgtcagttgcatgtcgctgcgcttctga

SEQ ID NO: 8 - protein, 254 aa, P. aeruginosa
MFKHIIARTPARSLVDGLTSSHLGKPDYAKALEQHNAYIRALQTCDVDITLLPPDERFPDSVFVEDPVLCTSRCAIITRPGAESRRGETEIIEETVQRFYPGKVERIEAPGTVEAGDIMMVGDHFYIGESARTNAEGARQMIAILEKHGLSGSVVRLEKVLHLKTGLAYLEHNNLLAAGEFVSKPEFQDFNIIEIPEEESYAANCIWVNERVIMPAGYPRTREKIARLGYRVIEVDTSEYRKIDGGVSCMSLRF

SEQ ID NO: 9 - DNA, 1257 bp, P. aeruginosa
  CDS (1)..(1257)
atgagcacggaaaaaaccaaacttggcgtccactccgaagccggcaaactgcgcaaagtgatggtctgctcgcccggactcgcccaccagcgcctgaccccgagcaactgcgacgagttgctgttcgacgacgtgatctgggtgaaccaggccaagcgcgaccacttcgacttcgtcaccaagatgcgcgagcgcggcatcgacgtcctcgagatgcacaatctgctgaccgagaccatccagaacccggaagcgctgaagtggatcctcgatcgcaagatcaccgccgacagcgtcggcctgggcctgaccagcgagctgcgctcctggctggagagcctggagccgcgcaagctggccgagtacctgatcggcggcgtcgccgctgacgacctgcccgccagcgaaggcgccaacatcctcaagatgtaccgcgagtacctgggccattccagcttcctgctgccgccgttgccgaacacccagttcacccgcgacaccacttgctggatctacggcggcgtgaccctgaacccgatgtactggccggcgcgacgacaggaaaccctgctgaccaccgccatctacaagttccaccccgagttcgccaacgccgagttcgagatctggtacggcgacccggacaaggaccacggctcctcgaccctggaaggcggcgacgtgatgccgatcggcaacggcgtggtcctgatcggcatgggcgagcgctcctcgcgccaggccatcggtcaggtcgcccagtcgctgttcgccaagggcgccgccgagcgggtgatcgtcgccggcctgccgaagtcccgcgccgcgatgcacctggacaccgtgttcagcttctgcgaccgcgacctggtcacggtcttcccggaagtggtcaaggaaatcgtgcccttcagcctgcgccccgatccgagcagcccctacggcatgaacatccgccgcgaggagaaaaccttcctcgaagtggtcgccgaatccctcggcctgaagaaactgcgcgtggtcgagaccggcggcaacagcttcgccgccgagcgcgagcaatgggacgacggtaacaacgtggtctgcctggagccgggcgtggtggtcggctacgaccgcaacacctacaccaacaccctgctgcgcaaggccggcgtcgaggtcatcaccatcagcgccagcgaactgggtcgcggtcgcggcggcggccactgcatgacctgcccgatcgtccgcgacccgatcgactactga

SEQ ID NO: 10 - protein, 418 aa, P. aeruginosa
MSTEKTKLGVHSEAGKLRKVMVCSPGLAHQRLTPSNCDELLFDDVIWVNQAKRDHFDFVTKMRERGIDVLEMHNLLTETIQNPEALKWILDRKITADSVGLGLTSELRSWLESLEPRKLAEYLIGGVAADDLPASEGANILKMYREYLGHSSFLLPPLPNTQFTRDTTCWIYGGVTLNPMYWPARRQETLLTTAIYKFHPEFANAEFEIWYGDPDKDHGSSTLEGGDVMPIGNGVVLIGMGERSSRQAIGQVAQSLFAKGAAERVIVAGLPKSRAAMHLDTVFSFCDRDLVTVFPEVVKEIVPFSLRPDPSSPYGMNIRREEKTFLEVVAESLGLKKLRVVETGGNSFAAEREQWDDGNNVVCLEPGVVVGYDRNTYTNTLLRKAGVEVITISASELGRGRGGGHCMTCPIVRDPIDY

SEQ ID NO: 11 - DNA, 1014 bp, M. tuberculosis
  CDS (69)..(986)
atgtatcaatggaaaatacgcaacgaccatcgtttgattgtgaaatcagagccaaatatcgttggtttatgacggattcctacgtcgctgctgcccgtctagggtcacctgcacgccgcaccccccggacgcggcggtatgcaatgaccccgccggccttctttgccgtcgcatacgcgatcaacccctggatggacgtcaccgcgccagtcgacgtccaagtcgcgcaagcacagtgggagcacctccaccagacctatcttcggctaggccacagcgtggatctgatcgagcccatttccgggttaccggacatggtgtacaccgccaacggtgggttcatcgcgcacgacatcgccgtggtcgcccggttccggttccccgaacgagctggtgagtctagagcctatgccagctggatgtcctcggtcggatatcgcccggtgaccacccgccacgtcaacgagggacagggcgacctgctgatggttggcgaaagggtgttggcgggctacggctttcgcacagaccagcgcgcacacgccgaaatcgccgcggtgcttggtctgccggtggtctccctcgagttggtcgacccacggttctatcacctggacaccgcgctggccgtgctcgacgaccacacgatcgcctactacccgccggcgttcagtacggcagcgcaggaacagttgtcggcgctgttccccgacgcgattgtggtcggcagtgccgacgcgttcgtgttcggactcaacgccgtctctgacggtctgaacgtagtgcttccggtcgcggccatgggttttgcggcgcagttacgcgcagccggcttcgagccggtcggtgtcgatctgtccgagctgctcaagggcggcggttccgtcaagtgctgcacgctggagatacacccatgacaaatctcgcggatgccactcaggccactatggcactggtcgaaaggcatgcagcgcacaattattcgccgctgcctgtggtggcggccagcgctga

SEQ ID NO: 12 - protein, 282 aa, M. tuberculosis
MTDSYVAAARLGSPARRTPRTRRYAMTPPAFFAVAYAINPWMDVTAPVDVQVAQAQWEHLHQTYLRLGHSVDLIEPISGLPDMVYTANGGFIAHDIAVVARFRFPERAGESRAYASWMSSVGYRPVTTRHVNEGQGDLLMVGERVLAGYGFRTDQRAHAEIAAVLGLPVVSLELVDPRFYHLDTALAVLDDHTIAYYPPAFSTAAQEQLSALFPDAIVVGSADAFVFGLNAVSDGLNVVLPVAAMGFAAQLRAAGFEPVGVDLSELLKGGGSVKCCTLEIHP

SEQ ID NO: 13 - protein, 305 aa, M. tuberculosis
NVSMENTQRPSFDCEIRAKYRWFMTDSYVAAARLGSPARRTPRTRRYAMTPPAFFAVAYAINPWMDVTAPVDVQVAQAQWEHLHQTYLRLGHSVDLIEPISGLPDMVYTANGGFIAHDIAVVARFRFPERAGESRAYASWMSSVGYRPVTTRHVNEGQGDLLMVGERVLAGYGFRTDQRAHAEIAAVLGLPVVSLELVDPRFYHLDTALAVLDDHTIAYYPPAFSTAAQEQLSALFPDAIVVGSADAFVFGLNAVSDGLNVVLPVAAMGFAAQLRAAGFEPVGVDLSELLKGGGSVKCCTLEIHP

(19) United States
(12) Patent Application Publication
(10) Pub. No.: US 2005/0153315 A1
(43) Pub. Date: Jul. 14, 2005

(54) Dimethylarginine dimethylaminohydrolases

(75) Inventors: Patrick John Thompson Vallance, London (GB); Ian George Charles, London (GB); James Mitchell Leiper, London (GB); Guy St. John Whitley, London (GB)

Correspondence Address: NIXON & VANDERHYE, PC, 1100 N GLEBE ROAD, 8TH FLOOR, ARLINGTON, VA, 22201-4714, US

(73) Assignee: UNIVERSITY COLLEGE LONDON, London (GB)

(21) Appl. No.: 10/966,039

(22) Filed: Oct. 18, 2004

Related U.S. Application Data
(62) Division of application No. 09/889,733, filed on Sep. 14, 2001, now abandoned.

(30) Foreign Application Priority Data
Jan. 26, 1999 (GB) 9901705.5
Jun. 4, 1999 (GB) 9913066.8

(51) Int. Cl. C12Q 1/68; C07H 21/04; A61K 39/395; C12N 9/90

(52) U.S. Cl. 435/6; 435/69.1; 435/233; 435/320.1; 435/325; 530/388.26; 424/146.1; 536/23.2

(57) ABSTRACT

Two dimethylarginine dimethylaminohydrolase (DDAH) genes have been cloned from humans. These genes can be used to screen for inhibitors and activators of activity and/or expression of DDAHs. Inhibitors and activators of activity and/or expression of DDAHs are useful in the treatment of conditions in which abnormal metabolism of nitric oxide is implicated.

### TECHNICAL FIELD OF THE INVENTION

This invention relates to methods of screening for compounds which specifically regulate different isoforms of dimethylarginine dimethylaminohydrolase.

### BACKGROUND OF THE INVENTION

Arginine residues in proteins are methylated by a family of Protein arginine N-methyltransferases (PRMTs). These enzymes catalyze the methylation of guanidino nitrogens of arginine to produce N^{G} monomethyl-L-arginine (L-NMMA), N^{G}N′^{G} dimethyl-L-arginine (asymmetric dimethylarginine; ADMA) and N^{G}N^{G} dimethylarginine (symmetric dimethylarginine; SDMA). Proteolysis of proteins containing these residues releases free methylarginines. Although the biological role of methylarginine residues is unclear, free L-NMMA and ADMA, but not SDMA, are inhibitors of all three isoforms of nitric oxide synthase (NOS) and might alter NOS activity in health or disease.

Free methylarginines are found in cell cytosol, plasma and tissues and their concentrations differ between tissues and between regions within a single tissue or organ. Elevated concentrations of ADMA have been detected in endothelial cells repopulating blood vessels damaged by balloon injury, in the plasma of patients or experimental animals with hyperlipidaemia, renal failure or athersclerosis, and in patients with schizophrenia or multiple sclerosis. Altered biosynthesis of nitric oxide (NO) has been implicated in the pathogenesis of all of these conditions and it is possible that the accumulation of endogenous ADMA underlies the inhibition of NO generation.

The production of methylarginines is probably an obligatory step in protein turnover, and rates of production may show tissue specific and temporal variations. However, L-NMMA and ADMA, but not SDMA, are actively metabolised to citrulline and methylamines by the action of dimethylarginine dimethylaminohydrolase (DDAH). Certain tissues which express NOSs also appear to express DDAH. Pharmacological inhibition of DDAH increases the concentration of ADMA in endothelial cells and inhibits NO-mediated endothelium-dependent relaxation of blood vessels. These observations suggest that DDAH activity ensures that the local concentration of ADMA does not normally rise sufficiently to affect NO generation, and that changes in DDAH activity could actively alter NOS activity.

### SUMMARY OF THE INVENTION

The present invention is based on our finding that humans express two functionally active methylarginases, which we have called DDAHI and DDAHII. We have cloned the polynucleotides that encode DDAHI and DDAHII isoforms and have studied the expression patterns of these two methylarginases via RNA blotting. These experiments revealed that DDAHI has a tissue distribution in humans which is similar to that of the neuronal isoform of nitric oxide synthase (nNOS), whilst DDAHII is highly expressed in vascular tissues which also express endothelial (eNOS).

Furthermore, we have shown that DDAHII is expressed in immune tissues and is located to chromosome 6p21.3. That chromosomal locus has been implicated in susceptibility to several diseases, including rheumatoid arthritis and insulin-dependent diabetes mellitus (IDDM) and the altered production of nitric oxide has been implicated in the pathology of both of those diseases.

Our data provide evidence that methylarginine concentration is actively regulated in cells that express NOS and further, suggest that there is a mechanism of regulation of NOS whereby different isoforms of NOS are specifically regulated as methylarginine concentrations are modulated by the action of specific DDAH enzymes.

DDAHI and DDAHII therefore provide new targets for the isolation of substances which can specifically modulate the activity of particular NOS isoforms or other arginine utilising enzymes through specific interaction with particular DDAH isoforms. Such substances may be useful in the treatment of diseases in which abnormal levels of methylarginines and/or nitric oxide are implicated.

Furthermore, we have found that the human DDAHI and DDAHII share significant homology with bacterial arginine deiminases. Arginine deiminases have only been described in prokaryotic organisms and the primitive eukaryote Giardia intestinalis. Arginine deiminases catalyse the hydrolysis of arginine to ammonia and citrulline in a reaction that closely resembles the hydrolysis of methylarginine to methylamine and citrulline catalysed by DDAHI.

We have isolated DDAHI sequences from three species of bacteria and an arginine deiminase sequence from P. aeruginosa. The enzymes encoded by these sequences can be expressed at high levels and large quantities of the expressed enzyme can be recovered. Thus we have identified an excellent source of enzymes which can be used to identify compounds capable of modulating the activity of DDAH enzymes.

According to the present invention there is thus provided a polynucleotide which:
(a) encodes a polypeptide that has the properties of a methylarginase, which polynucleotide is selected from:
(1) the coding sequence of SEQ ID NO: 1, 3, 5, 7, 9 or 11;
(2) a sequence which hybridises selectively to the complement of a sequence defined in (1); and
(3) a sequence that is degenerate as a result of the genetic code with respect to a sequence defined in (1) or (2); or
(b) is a sequence complementary to a polynucleotide defined in (a).

The invention also provides:
a polypeptide which has methylarginase activity and which comprises the sequence set out in SEQ ID NO: 2, 4, 6, 8, 10 or 12, a sequence substantially homologous thereto or a fragment of either said sequence.
a vector incorporating a polynucleotide of the invention.
a cell harbouring a polynucleotide, a peptide or a vector of the invention.
an antibody capable of binding a polypeptide encoded by a polynucleotide of the invention or a polypeptide of the invention.
a non-human animal which is not capable of expressing or is not capable of expressing an active form of one or more isoforms of methylarginase.
a process for the preparation of a polypeptide which has methylarginase activity, which process comprises cultivating a host cell harbouring an expression vector of the invention under conditions to provide for expression of the said polypeptide, and recovering the expressed polypeptide.
a modulator of methylarginase activity.
a method for identifying a modulator of methylarginase activity and/or expression, comprising:
   (i) contacting a polynucleotide of the invention, a polypeptide of the invention, a vector of the invention or a cell of the invention and a test substance under conditions that would permit methylarginase activity in the absence of the test substance; and
   (ii) determining thereby whether the said substance modulates the activity and/or expression of methylarginase.
a modulator of methylarginase activity and/or expression identified by the method of the method of the invention.
a polynucleotide, a polypeptide, an expression vector or a modulator of the invention for use in a method of treatment of the human or animal body by therapy.
use of a polynucleotide, a polypeptide, an expression vector or a modulator which is an activator of the invention for the manufacture of a medicament for use in the treatment of hyperlipidaemia, renal failure, hypertension, restenosis after angioplasty, atherosclerosis, complications of heart failure, schizophrenia, multiple sclerosis or cancer.
use of a modulator of the invention which is an inhibitor of methylarginase activity and/or expression for the manufacture of a medicament for use in the treatment of ischeamia-reperfusion injury of the brain or heart, cancer, lethal hypotension in severe inflammatory conditions such as septic shock or multi-organ failure, or local and systemic inflammatory disorders including arthritis, skin disorders, inflammatory cardiac disease or migraine.
a pharmaceutical composition comprising a polynucleotide, a polypeptide, an expression vector or a modulator which is an activator of the invention and a pharmaceutically acceptable carrier and/or diluent.
a method of treating a human or animal suffering from hyperlipidaemia, renal failure, hypertension, restenosis after angioplasty, atherosclerosis, complications of heart failure, schizophrenia, multiple sclerosis or cancer, which method comprises administering to the host a therapeutically effective amount of a polypeptide, an expression vector, or a modulator which is an activator of the invention.
a method of treating a human or animal suffering from ischeamia-reperfusion injury of the brain or heart, cancer, lethal hypotension in severe inflammatory conditions such as septic shock or multi-organ failure, or local and systemic inflammatory disorders including arthritis, skin disorders, inflammatory cardiac disease or migraine, which method comprises administering to the host a therapeutically effective amount of a modulator which is an inhibitor of the invention.
products containing a modulator which is an inhibitor of the invention and a methylarginine as a combined preparation for simultaneous, separate or sequential use in a method of treatment of ischeamia-reperfusion injury of the brain or heart, cancer, lethal hypotension in severe inflammatory conditions such as septic shock or multi-organ failure, or local and systemic inflammatory disorders including arthritis, skin disorders, inflammatory cardiac disease or migraine

### BRIEF DESCRIPTION OF THE DRAWINGS

FIG. 1 shows an amino acid alignment of rat and human DDAHI with human DDAHII. The derived amino acid sequences of human and rat DDAHI and human DDAHII were aligned using the clustal programme. Amino acid identities are indicated (*), highly conservative substitutions (:) and conservative substitutions (.).

FIG. 2 shows recombinant expression of human DDAH II. Aliquots of E. coli transfected with either empty vector (lanes 1 and 3) or vector containing human DDAH II cDNA (lanes 2 and 4) were resolved on 15% SDS-PAGE gels. Gels were either stained for total protein with coomassie blue (lanes 1 and 2) or processed for western blotting (lanes 3 and 4) as described under Experimental Procedures. The filled arrow indicates the ˜40 kDa recombinant protein that is specifically recognised by the anti-PentaHis antibody. The migration of molecular weight markers is indicated.

FIG. 3 shows DDAH activity of recombinant DDAH II. Aliquots of cell lysates of E. coli transfected with either empty vector or vector containing human DDAH II cDNA were assayed for DDAH activity as described under Experimental Procedures. Assays were performed in triplicate and the data is expressed as the average of the three replicates after subtraction of background. The data presented is the result of one representative experiment. Similar results were obtained in four independent experiments. The data shown represent the hydrolysis of ˜1 mmol L-NMMA hr⁻¹ by E. coli lysates containing recombinant DDAH II. Under the same conditions, a 30% rat liver homogenate hydrolysed ˜18 mmol L-NMMA hr−1

FIG. 4 shows tissue distribution of human DDAH and NOS isoforms. Labelled probes specific for human DDAH I, DDAH II, neuronal NOS, endothelial NOS and b-actin were sequentially hybridized to a commercially availible multiple-tissue northern blot. The migration of molecular weight markers is indicated.

FIG. 5 shows aligment of human DDAHI and II with Pseudomonas Arginine Deiminase. The derived amino acid of human DDAHI and II were aligned with the amino acid sequence of Pseudomonas X arginine deiminase. Amino acid identities are indicated (*), highly conservative substitutions (:) and conservative substitutions (.). Boxed regions indicated motifs highly conserved between arginine deiminases.

FIG. 6A shows the alignment using ClustalW of human DDAH I and DDAHs from S.coelicolor, P.aeruginosa and M.tuberculosis. Identical amino acids are indicated by (*), highly conserved amino acid substitutions by (:) and conserved amino acid substitutions by (.).

S. coelicolor DDAH is encoded by residues 33784 to 33011 of cosmid St4C6. The sequence does not have an individual accession number. P. aeruginosa DDAH sequence is contained within a contiguous genomic DNA sequence (contig 1281). Again, the sequence does not have an individual accession number. M. tuberculosis DDAH has been deposited under accession number DDAH Z797022.

FIG. 6B shows a similar alignment using ClustalW of P. aeruginosa DDAH and arginine deiminase.

FIG. 7 shows enzymatic activity of ScDDAH and PaDDAH. The effect of 10 mM ADMA and SDMA on recombinant ScDDAH and paDDAH was studied using the assay conditions described in the material and methods section below. Assays were carried out in triplicate on aliquots of cell lysates containing empty vector, scDDAH cDNA or paDDAH cDNA and data was expressed as a mean of the total number of replicates after subtraction of background. The results shown are the mean of three independent experiments.

### DETAILED DESCRIPTION OF THE INVENTION

Polynucleotides

The invention provides a polynucleotide which:
(a) encodes a polypeptide that has the properties of a methylarginase, which polynucleotide is selected from:
(1) the coding sequence of SEQ ID NO: 1, 3, 5, 7, 9 or 11;
(2) a sequence which hybridises selectively to the complement of a sequence defined in (1); and
(3) a sequence that is degenerate as a result of the genetic code with respect to a nucleic sequence defined in (1) or (2); or
(b) is a sequence complementary to a polynucleotide defined in (a).

SEQ ID NOS: 1, 3, 5, 7, 9 and 11 set out the sequences of human DDAHI, DDAHII, S. coelicolor DDAH, P. aeruginosa DDAH, P. aeruginosa arginine deiminase and M. tuberculosis DDAH respectively.

Polynucleotides of the invention also include variants of the coding sequence of SEQ ID NO: 1, 3, 5, 7, 9 or 11 which can function as methylarginases. Such variants thus have the ability to catalyze the production of citrulline from methylarginines. Typically a polynucleotide of the invention comprises a contiguous sequence of nucleotides which is capable of hybridizing under selective conditions to the complement of the coding sequence of SEQ ID NO: 1, 3, 5, 7, 9 or 11.

A polynucleotide of the invention and the complement of the coding sequence of SEQ ID NO: 1, 3, 5, 7, 9 or 11 can hydridize at a level significantly above background. Background hybridization may occur, for example, because of other cDNAs present in a cDNA library. The signal level generated by the interaction between a polynucleotide of the invention and the complement of the coding sequence of SEQ ID NO: 1, 3, 5, 7, 9 or 11 is typically at least 10 fold, preferably at least 100 fold, as intense as interactions between other polynucleotides and the coding sequence of SEQ ID NO: 1, 3, 5, 7, 9 or 11. The intensity of interaction may be measured, for example, by radiolabelling the probe, e.g. with ³²P. Selective hybridisation may typically be achieved using conditions of low stringency (0.03M sodium chloride and 0.03M sodium citrate at about 40° C.), medium stringency (for example, 0.03M sodium chloride and 0.03M sodium citrate at about 50° C.) or high stringency (for example, 0.03M sodium chloride and 0.03M sodium citrate at about 60° C.).

A nucleotide sequence which is capable of selectively hybridizing to the complement of the DNA coding sequence of SEQ ID NOS: 1, 3, 5, 7, 9 or 11 will generally have at least 60%, at least 70%, at least 80%, at least 90%, at least 95%, at least 98% or at least 99% sequence identity to the coding sequence of SEQ ID NO: 1, 3, 5, 7, 9 or 11 over a region of at least 20, preferably at least 30, for instance at least 40, at least 60, more preferably at least 100 contiguous nucleotides or most preferably over the full length of SEQ ID NO: 1, 3, 5, 7, 9 or 11.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The coding sequence of SEQ ID NO: 1, 3, 5, 7, 9 or 11 may be modified by nucleotide substitutions, for example from 1, 2 or 3 to 10, 25, 50 or 100 substitutions. The polynucleotide of SEQ ID NO: 1, 3, 5, 7, 9 or 11 may alternatively or additionally be modified by one or more insertions and/or deletions and/or by an extension at either or both ends. The modified polynucleotide generally encodes a polypeptide which has methylarginase activity. Degenerate substitutions may be made and/or substitutions may be made which would result in a conservative amino acid substitution when the modified sequence is translated, for example as shown in the Table below.

Polynucleotides of the invention may comprise DNA or RNA. They may also be polynucleotides which include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or lifespan of polynucleotides of the invention.

Polynucleotides of the invention may be used as a primer, e.g. a PCR primer, a primer for an alternative amplification reaction, a probe e.g. labeled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO: 1, 3, 5, 7, 9 or 11.

Polynucleotides such as a DNA polynucleotide and primers according to the invention may be produced recombinantly, synthetically, or by any means available to those of skill in the art. They may also be cloned by standard techniques. The polynucleotides are typically provided in isolated and/or purified form.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15-30 nucleotides) to a region of the G14 gene which it is desired to clone, bringing the primers into contact with mRNA or cDNA obtained from a human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Such techniques may be used to obtain all or part of the DDAHI and DDAHII genes described herein. Genomic clones corresponding to the cDNA of SEQ ID NOS: 1, 3, 5, 7, 9 or 11 containing, for example, introns and promoter regions are also aspects of the invention and may also be produced using recombinant means, for example using PCR (polymerase chain reaction) cloning techniques, starting with genomic DNA from for example a bacterial, an animal or a human cell.

Although in general the techniques mentioned herein are well known in the art, reference may be made in particular to Sambrook et al, 1989, Molecular Cloning: a laboratory manual.

Polynucleotides which do not have 100% sequence identity to the sequence of SEQ ID NOS: 1, 3, 5, 7, 9 or 11 but fall within the scope of the invention can be obtained in a number of ways:

1. Other human allelic variants of the human DDAHI and DDAHII sequences given in SEQ ID NOS: 1 and 3 may be obtained for example by probing genomic DNA libraries made from a range of individuals, for example individuals from different populations, or individuals with different types of disorder related to aberrant NO metabolism, using probes as described above.

In addition, homologues of SEQ ID NO: 1, 3, 5, 7, 9 or 11 may be obtained from other animals particularly mammals (for example mice and rabbits) or fish (for example Fugu) or insects (for example D. melanogaster) or other invertebrates (for example C. elegans), plants (for example A. thaliana), bacteria and yeasts and such homologues and fragments thereof in general will be capable of selectively hybridising to the coding sequence of SEQ ID NOS: 1 and 3 or its complement. Such sequences may be obtained by probing cDNA or genomic libraries from dividing cells or tissues or other animal species with probes as described above. Degenerate probes can be prepared by means known in the art to take into account the possibility of degenerate variation between the DNA sequence of SEQ ID NOS: 1, 3, 7, 9 or 11 and the sequences being probed for under the selective hybridization conditions given above.

2. Allelic variants and species homologues may also be obtained using degenerate PCR which will use primers designed to target sequences within the variants and homologues encoding likely conserved amino acid sequences. Likely conserved sequences can be predicted from aligning the amino acid sequences of the invention. The primers will contain one or more degenerate positions and will be used at stringency conditions lower than those used for cloning sequences with single sequence primers against known sequences.

3. Alternatively, polynucleotides may be obtained by site directed mutagenesis of SEQ ID NO: 1, 3, 5, 7, 9 or 11 or allelic variants thereof. This may be useful where, for example, silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The invention further provides double stranded polynucleotides comprising a polynucleotide of the invention and its complement.

Polynucleotides, probes or primers of the invention may carry a revealing label. Suitable labels include radioisotopes such as ³²P or ³⁵S, enzyme labels, or other protein labels such as biotin. Such labels may be added to polynucleotides, probes or primers of the invention and may be detected using techniques known per se.

Polypeptides

A polypeptide of the invention comprises the amino acid sequence set out in SEQ ID NO: 2, 4, 6, 8, 10 or 12 or a substantially homologous sequence, or a fragment of either said sequence and has methylarginase activity. In general, the naturally occurring amino acid sequence shown in SEQ ID NO: 2, 4, 6, 8, 10 or 12 is preferred.

SEQ ID NOS: 2, 4, 6, 8, 10 and 12 set out the amino acid sequences of human DDAHI, human DDAHII, S. coelicolor DDAH. P.aeruginosa DDAH, P.aeruginosa arginine deiminase and M.tuberculosis DDAH respectively

In particular, a polypeptide of the invention may comprise:
(a) the polypeptide sequence of SEQ ID NO: 2, 4, 6, 8, 10 or 12;
(b) an allelic variant or species homologue thereof; or
(c) a protein with at least 70, at least 80, at least 90, at least 95, at least 98 or at least 99% sequence identity to (a) or (b).

An allelic variant will be a variant which will occur naturally, for example, in a human, bacterium or yeast and which will function in a substantially similar manner to the protein of SEQ ID NO: 2, 4, 6, 8, 10 or 12, for example it acts as a methylarginase. Similarly, a species homologue of the protein will be the equivalent protein which occurs naturally in another species and which can function as a methylarginase.

Allelic variants and species homologues can be obtained by following the procedures described herein for the production of the polypeptides of SEQ ID NO: 2, 4, 6, 8, 10 or 12 and performing such procedures on a suitable cell source e.g. a human or bacterium cell. It will also be possible to use a probe as defined above to probe libraries made from human or bacterial cells in order to obtain clones encoding the allelic or species variants. The clones can be manipulated by conventional techniques to generate a polypeptide of the invention which can then be produced by recombinant or synthetic techniques known per se.

A polypeptide of the invention preferably has at least 60% sequence identity to the protein of SEQ ID NO: 3, more preferably at least 70%, at least 80%, at least 90%, at least 95%, at least 97% or at least 99% sequence identity thereto over a region of at least 20, preferably at least 30, for instance at least 40, at least 60, at least 100 contiguous amino acids or over over the full length of SEQ ID NO: 2, 4, 6, 8, 10 or 12.

The sequence of the polypeptide of SEQ ID NO: 2, 4, 6, 8, 10 or 12 and of allelic variants and species homologues can thus be modified to provide polypeptides of the invention. Amino acid substitutions may be made, for example from 1, 2 or 3 to 10, 20 or 30 substitutions. The modified polypeptide generally retains activity as a methylarginase, as defined herein. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |

| | |
|---|---|
| AROMATIC | H F W Y |

Polypeptides of the invention also include fragments of the above-mentioned full length polypeptides and variants thereof, including fragments of the sequence set out in SEQ ID NO: 2, 4, 6, 8, 10 or 12. Such fragments typically retain activity as a methylarginase.

Other preferred fragments include those which include an epitope. Suitable fragments will be at least 5, e.g. at least 10, at least 12, at least 15 or at least 20 amino acids in size. Epitope fragments may typically be up to 50, 60, 70, 80, 100, 150 or 200 amino acids in size. Polypeptide fragments of the polypeptides of SEQ ID NO: 3, and allelic and species variants thereof may contain one or more (e.g. 1, 2, 3 or 5 to 10, 20 or 30) substitutions, deletions or insertions, including conservative substitutions. Epitopes may be determined by techniques such as peptide scanning techniques already known in the art. These fragments will be useful for obtaining antibodies to polypeptides of the invention.

Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 50%. e.g. more than 80%, 90%, 95% or 99%, by weight of the polypeptide in the preparation is a polypeptide of the invention.

Polypeptides of the invention may be chemically modified, e.g. post-translationally modified. For example, they may be glycosylated or comprise modified amino acid residues. They may also be modified by the addition of Histidine residues to assist their purification or by the addition of a signal sequence to promote their secretion from a cell. Such modified polypeptides and proteins fall within the scope of the term “polypeptide” of the invention.

Polypeptides of the invention may be modified for example by the addition of Histidine residues or a T7 tag to assist their identification or purification or by the addition of a signal sequence to promoter their secretion from a cell where the polypeptide does not naturally contain such a sequence.

Vectors

Polynucleotides of the invention can be incorporated into a recombinant replicable vector. The vector may be used to replicate the nucleic acid in a compatible host cell. Thus, in a further embodiment, the invention provides a method of making polypeptides of the invention by introducing a polynucleotide of the invention into a replicable vector, introducing the vector into a compatible host cell, and growing the host cell under conditions which bring about replication of the vector. The vector may be recovered from the host cell.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used in a method of controlling the levels of methylarginases or their variants or species homologues.

Preferably, a polynucleotide of the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

Vectors of the invention may be transformed into a suitable host cell as described above to provide for expression of a polypeptide of the invention. Thus, in a further aspect the invention provides a process for preparing a polypeptide according to the invention which comprises cultivating a host cell transformed or transfected with an expression vector encoding the polypeptide, and recovering the expressed polypeptide.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistence gene in the case of a bacterial plasmid or a resistance gene for a fungal vector. Vectors may be used in vitro, for example for the production of RNA or used to transfect or transform a host cell, for example, E. coli. The vectors may also be adapted to be used in vivo, for example in a method of gene therapy.

A further embodiment of the invention provides host cells transformed or transfected with the vectors for the replication and/or expression of polynucleotides of the invention. The cells will be chosen to be compatible with the said vector and may for example baterial (eg. E. coli), yeast, insect or mammalian.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters, in particular endothelial or neuronal cell specific promoters (for example the DDAHI and DDAHII promoters), are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to antisense RNA which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors (for example as disclosed in WO 98/04726 and WO 98/30707) and retroviruses, including lentiviruses, adenoviruses, adeno-associated viruses and HPV viruses (such as HPV-16 or HPV-18). Gene transfer techniques using these viruses are known to those skilled in the art. Retrovirus vectors for example may be used to stably integrate the polynucleotide giving rise to the antisense RNA into the host genome. Replication-defective adenovirus vectors by contrast remain episomal and therefore allow transient expression.

Antibodies

The invention also provides monoclonal or polyclonal antibodies to polypeptides encoded by polynucleotides of the invention, to polypeptides of the invention and to fragments thereof. The invention further provides a process for the production of monoclonal or polyclonal antibodies of the invention.

Antibodies of the invention may be antibodies to human polypeptides or fragments thereof. Preferred antibodies are those which are able to discriminate between a DDAHI or polypeptide or fragment thereof and a DDAHII polypeptide or fragment thereof, and in particular between the human DDAHI and II polypeptides and fragments thereof.

For the purposes of this invention, the term “antibody”, unless specified to the contrary, includes fragments of whole antibodies which maintain their binding activity for a polypeptide encoded by a polynucleotide of the invention, a polypeptide of the invention or a fragment thereof. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies of the invention may be used, inter alia, in a method for detecting polypeptides of the invention present in a biological sample which method comprises
(a) providing an antibody of the invention;
(b) incubating a biological sample with said antibody under conditions which allow for the formation of an antibody-antigen complex; and
(c) determining whether antibody-antigen complex comprising said antibody is formed.

A sample may be for example a tissue extract. Antibodies of the invention may be bound to a solid support and/or packaged into kits in a suitable container along with suitable reagents, controls, instructions, etc.

Antibodies of the invention can be produced by any suitable method. Means for preparing and characterising antibodies are well known in the art, see for example Harlow and Lane (1988) “Antibodies: A Laboratory Manual”, cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

Animals

The invention provides a non-human animal which is not capable of expressing or is not capable of expressing an active form of one or more isoforms of methylarginase. Preferably the methylarginase isoform is a DDAHI and/or a DDAH II. Typically, the wild-type of the non-human animal will be capable of expressing an active form of the methylarginase which the non-human animal of the invention cannot.

An animal which is not capable of expressing one or more isoforms of methylarginase is one which shows substantially no detectable expression of at least one methylarginase mRNA. An animal which is not capable of expressing an active form of one or more isoforms of methylarginase is one which expresses at least one methylarginase related polypeptide, which polypeptide shows substantially no methylarginase activity.

An animal of the invention may be one in which polynucleotide sequence from a methylarginase encoding gene locus has been deleted or replaced with polynucleotide sequences from another locus or from another organism. Thus, substantially no methylarginase mRNA may be expressed from that methylarginase locus. Alternatively, the coding sequence of a methylarginase gene may have been altered such that the expressed polypeptide shows substantially no methylarginase activity.

Typically a non-human animal of the invention is a so-called “knock-out animal”. The term “knock-out animal” is well known to those skilled in the art. Typically, a non-human animal of the invention, for example a knock-out animal, will be a transgenic animal.

A knock-out animal can be produced according to any suitable method. In general, a polynucleotide construct is produced comprising a marker gene, for example, flanked by genomic sequences. Those genomic sequences correspond to genomic sequences at the methylarginase encoding gene locus of the animal in question. Thus, if the polynucleotide construct is contacted with the methylarginase encoding gene locus of the animal of interest, homologous recombination events may lead to replacement of the chromosomal sequence bordered by the genomic sequences used in the polynucleotide construct with the marker gene. If the marker gene replaces coding sequence or a regulatory sequence, for example a promoter sequence, gene expression and/or activity may be abolished.

The polynucleotide construct is typically transferred into a fertilized egg by pronuclear microinjection so that the contacting described above can occur. Alternative approaches may be used for example, embryonic stem cells or retroviral mediated gene transfer into germ lines. Whichever approach is taken, transgenic animals are then generated. For example, microinjected eggs may be implanted into a host female and the progeny may be screened for the expression of the marker gene. The founder animals that are obtained may be bred.

A non-human animal of the invention may be for example a mammal, preferably a mouse, a reptile, an amphibian, a bird or a fish. Preferred non-human animals are mice. Preferred animals are thus mice in which all or part of the DDAHI or DDAHII gene locus has been deleted or replaced for example, ie. DDAHI or DDAHII knock-out mice.

The invention also provides a cell or cell-line derived from a non-human animal of the invention.

The transgenic technology described above is of course equally applicable to the production of non-human animals which over-express one or more isoforms of methylarginase, or express one or more isoforms of methylarginase which have unusually high activities. In such cases the polynucleotide construct used does not replace an endogenous portion of a methylarginase gene with a marker gene. Instead, an endogenous methylarginase gene may be replaced with a polynucleotide construct comprising a promoter, for example one which drives high levels of expression, operably lined to a methylarginase coding sequence. Alternatively, the construct may comprise a methylarginase promoter sequence operably linked to a reporter gene. It is also possible to produce constructs which do not replace endogenous methylarginase sequences. Use of such constructs will result in animals which contain endogenous methylarginase sequences and the sequences insert by the construct.

The non-human animals described above may be used in the screening assays described below for the identification of modulators of methylarginase activity and/or expression.

Assays

The invention provides a method for identifying a modulator of methylarginase activity and/or expression, comprising:
(i) contacting a polynucleotide according to the invention, a polypeptide according to the invention, a vector according to the invention or a cell according to the invention and a test substance under conditions that would permit methylarginase activity in the absence of the test substance; and
(ii) determining thereby whether the said substance modulates the activity and/or expression of methylarginase.

Any suitable assay format may be used for identifying a modulator of methylarginase activity and/or expression.

In the case of using a polynucleotide or vector of the invention, the assay will typically be carried out on a cell harbouring the polynucleotide or vector or on a cell extract comprising the polynucleotide or vector. The cell or cell extract will typically allow transcription and translation of the polynucleotide or vector in the absence of a test substance.

A typical assay is as follows:
a defined number of cells harbouring a polynucleotide or vector of the invention are inoculated in growth medium into the wells of a plastics micro-titre plate in the presence of a substance to be tested.
the micro-titre plates are covered and incubated at an appropriate temperature (eg. 37° C. for E. coli) in the dark.
samples are withdrawn at regular time intervals and assayed for methylarginase activity, as described in the Examples.
parallel control experiments can be carried out, in which the substance to be tested is omitted.

Also, as a control, the samples may be assayed for any other enzyme to exclude the possibility that the test substance is a general inhibitor of gene expression or enzyme activity.

The assay may also be carried out using a polypeptide of the invention, in which any suitable format may be used for identifying a modulator of methylarginase activity. Most preferably such an assay would be carried out in a single well of a plastics microtitre plate, so that high through-put screening for methylarginase activity modulators may be carried out. In practice, the enzyme reaction is commenced by addition of a methylarginase or a substrate for methylarginase. An assay for a methylarginase modulator may therefore be initiated by providing a medium, containing a test substance and one of a methylarginase and a methylarginase substrate. As a control, the progress of the assay can be followed in the absence of the test substance.

Also the substance tested may be tested with any other known polypeptide/enzyme to exclude the possibility that the test substance is a general inhibitor of enzyme activity.

Suitable methylarginases for the assay can be obtained using the recombinant techniques described above. Suitable substrates are those comprising asymmetric methylarginines, for example N^{G}monomethyl-L-arginine (L-NMMA), asymmetric dimethylarginine (ADMA). In addition to the methylarginase and a suitable substrate, the reaction mixture can contain a suitable buffer, suitable cofactors and suitable divalent cations as a cofactor. A suitable buffer includes any suitable biological buffer that can provide buffering capability at a pH conducive to the reaction requirements of the enzyme.

The assay of the invention may be carried out at any temperature at which a methylarginase, in the absence of any inhibitor, is active. Typically, however, the assay will be carried out in the range of from 25° C. to 37° C.

Measures of enzymatic activity of methylarginase activity are generally known to those skilled in the art, including equilibrium constants, reaction velocities of the appearance of reaction products or the consumption of reaction substrates, reaction kinetics, thermodynamics of reaction, spectrophotometric analysis of reaction products, detection of labelled reaction components, etc. See, generally, Segel, Biochemical Calculations, 2nd Edition, John Wiley and Sons, New York (1976); Suelter, A Practical Guide to Enzymology, John Eiley and Sons, New York (1985). The preferred method of measuring enzymatic activity is by measuring [¹⁴C]citrulline production after the methylarginase has been incubated with [¹⁴C]L-NMMA or [¹⁴C]ADMA.

Assays can also be carried out using constructs comprising a a methylarginase gene promoter operably linked to a heterologous coding sequence, to identify compounds which modulate expression of methylarginases at the transcriptional level.

A promoter means a transcriptional promoter. Methylarginase gene promoters can be isolated via methods known to those skilled in the art and as described above. The term “heterologous” indicates that the coding sequence is not operably linked to the promoter in nature; the coding sequence is generally from a different organism to the promoter.

The promoter sequence may be fused directly to a coding sequence or via a linker. The linker sequence may comprise an intron. Excluding the length of any intron sequence, the linker may be composed of up to 45 bases. The linker sequence may comprise a sequence having enhancer characteristics, to boost expression levels.

Preferably the promoter is operably linked to the coding sequence of a reporter polypeptide. The reporter polypeptide may be, for example, the bacterial polypeptide β-glucuronidase (GUS), green fluoresent protein (GFP), luciferase (luc), chloramphenicol transferase (CAT) or β-galactosidase (lacZ).

Promoter:reporter gene constructs such as those described above can be incorporated into a recombinant replicable vector. The vector may be used to replicate the nucleic acid construct in a compatible host cell. The vectors may be, for example, plasmid, virus or phage vectors provided with an origin of replication. Any host cell may be used in which the promoter is functional, but typically the host cell will be a cell of the species from which the promoter derives. The promoter:reporter gene constructs of the invention may be introduced into host cells using conventional techniques.

Thus the invention provides a method for identifying a modulator of methylarginase expression. Typically a promoter:reporter polypeptide construct or a cell harbouring that construct will be contacted with a test substance under conditions that would permit the expression of the reporter polypeptide in the absence of the test substance.

Any reporter polypeptide may be used, but typically GUS or GFP are used. GUS is assayed by measuring the hydrolysis of a suitable substrate, for example 5-bromo-4-chloro-3-indolyl-β-D-glucoronic acid (X-gluc) or 4-methylumbelliferyl-β-glucuronide (MUG). The hydrolysis of MUG yields a product which can be measured fluorometrically. GFP is quantified by measuring fluorescence at 590 nm after excitation at 494 nm. These methods are well known to those skilled in the art.

Methylarginases

Any methylarginase, for example a methylarginase encoded by a polynucleotide of the invention or a methylarginase having the amino acid sequence of a polypeptide of the invention, may be used in the assays described above. The enzymes may be prokaryotic or eukaryotic. They may be obtained from prokaryotic or eukaryotic extracts, for example from a microbial extract. Alternatively, the enzymes may be produced recombinantly, from, for example, bacteria, yeast or higher eukaryotic cells such as insect cell lines. Preferred methylarginases are a DDAHI, for example human DDAHI, or a DDAHI, for example a human DDAHII.

Recombinant expression of human DDAHII and bacterial DDAHI enzymes is described in the Examples.

Test Substances

A substance which modulates the expression or activity of a methylarginase may do so by binding directly to the relevant gene promoter, thus inhibiting or activating transcription of the gene. Inhibition may occur by preventing the initiation or completion of transcription. Activation may occur, for example, by increasing the affinity of the transcription complex for the promoter. Alternatively a modulator may bind to a protein which is associated with the promoter and is required for transcription.

A substance which modulates the activity of a methylarginase may do so by binding to the enzyme. Such binding may result in activation or inhibition of the protein.

Inhibition may occur, for example if the modulator resembles the substrate and binds at the active site of the methylarginase. The substrate is thus prevented from binding to the same active site and the rate of catalysis is reduced by reducing the proportion of enzyme molecules bound to substrate. A modulator which inhibits the activity of a methylarginase may do so by binding to the substrate. The modulator may itself catalyze a reaction of the substrate, so that the substrate is not available to the enzyme. Alternatively, the inhibitor may simply prevent the substrate binding to the enzyme.

Activation may occur, for example, if the modulator increases the affinity of the substrate for the enzyme or vice versa. This means that the proportion of enzyme molecules bound to a substrate is increased and the rate of catalysis will thus increase.

Suitable test substances which can be used in the assays described above include antibody products (for example, monoclonal and polyclonal antibodies, single chain antibodies, chimeric antibodies and CDR-grafted antibodies) which are specific for a methylarginase or mimics of a methylarginase. In addition, combinatorial libraries, defined chemical identities, peptide and peptide mimetics, oligonucleotides and natural product libraries, such as display libraries (e.g. phage display libraries) may also be tested. The candidate substances may be chemical compounds.

Batches of the test substances may be used in an initial screen of, for example, ten substances per reaction, and the substances of batches which show inhibition tested individually.

Modulators

A modulator of methylarginase, for example of a DDAH or an arginine deiminase, expression and/or activity is one which produces a measurable reduction or increase in methylarginase expression and/or activity in the assays described above. Thus, modulators of methylarginase expression and/or activity may be inhibitors or activators of methylarginase expression and/or activity. A modulator of a methylarginase may be a modulator of a DDAHI or a DDAHII.

Preferred inhibitors are those which inhibit methylarginase expression and/or activity by at least 10%, at least 20%, at least 30%, at least 40% at least 50%, at least 60%, at least 70%, at least 80%, at least 90%, at least 95% or at least 99% at a concentration of the inhibitor of 1 μg ml⁻¹, 10 μg ml⁻¹, 100 μg ml⁻¹, 500 μg ml⁻¹, 1 mg ml^{-1,} 10 mg ml⁻¹, 100 mg ml⁻¹.

Preferred activitors are those which activate methylarginase expression and/or activity by at least 10%, at least 25%, at least 50%, at least 100%, at least, 200%. at least 500% or at least 1000% at a concentration of the activator 1 μg ml⁻¹, 10 μg ml⁻¹, 100 μg ml ⁻¹, 500 μg ml⁻¹, 1 mg ml^{-1.} 10 mg ml⁻¹, 100 mg ml⁻¹.

The percentage inhibition or activation represents the percentage decrease or increase in expression/activity in a comparison of assays in the presence and absence of the test substance. Any combination of the above mentioned degrees of percentage inhibition or activation and concentration of inhibitor or activator may be used to define an inhibitor or activator of the invention, with greater inhibition or activation at lower concentrations being preferred.

Candidate substances which show activity in assays such as those described above can be tested in in vivo systems, an aninal model. Candidate inhibitors could be tested for their ability to increase ADMA and L-NMMA levels and/or to increase blood pressure and/or to decrease endothelium-dependent relaxation of blood vessels.

Candidate activators could be tested for their ability to increase nitric oxide generation as assessed by NOₓ measurement and/or to decrease levels of ADMA and L-NMMA. Ultimately such substances would be tested in animal models of the target disease states.

Therapeutic Use

Polynucleotides, peptides, expression vectors and modulators of methylarginase activity and/or expression and modulators of methylarginase activity and/or expression identified by the methods of the invention may be used for the treatment of a condition in which the abnormal metabolism of NO is implicated.

Polynucleotides peptides, expression vectors and activators of methylarginase activity and/or expression may be used in the treatment of conditions in which reduced NO production is implicated. In particular such conditions as hyperlipidaemia, renal failure, hypertension, restenosis after angioplasty, complications of heart failure, or atherosclerosis and its complications may be treated and patients with schizophrenia, multiple sclerosis or cancer may also be treated.

Modulators which are inhibitors of methylarginase activity and/or expression may be used in the treatment of conditions in which increased NO production is implicated. In particular conditions such as ischeamia-reperfusion injury of the brain or heart, cancer, lethal hypotension in severe inflammatory conditions such as septic shock or multi-organ failure, or local and systemic inflammatory disorders including arthritis, skin disorders, inflammatory cardiac disease or migraine may be treated.

Alternatively an inhibitor of methylarginase activity and/or expression could be used as a joint therapy together with an inhibitor of NOS activity (for example, a methylarginine). For example, a specific inhibitor of a DDAH isoform could be used with the methylarginine L-NMMA. This approach may radically alter the activity profile of L-NMMA and may result in L-NMMA having an increased inhibitory effect for a specific NOS isoform. Thus, the invention provides products containing an inhibitor of methylarginase activity and/or expression and a methylarginine as a combined preparation for simultaneous, separate or sequential use in the treatment of ischeamia-reperfusion injury of the brain or heart, cancer, lethal hypotension in severe inflammatory conditions such as septic shock or multi-organ failure, or local and systemic inflammatory disorders including arthritis, skin disorders, inflammatory cardiac disease or migraine.

Inhibitors of methylarginase expression and/or activity may also be used as antimicrobial, for example antibacterial, agents. Thus, inhibitors of microbial, for example bacterial, DDAH and arginine deiminase expression and/or activity are useful as antimicrobial agents. Therefore, the invention also provides an inhibitor of a bacterial methylarginase, for example a DDAH or an arginine deiminase, for use in the treatment of a bacterial infection.

A modulator of the invention may be administered in a variety of dosage forms. Thus, they can be administered orally, for example as tablets, troches, lozenges, aqueous or oily suspensions, dispersible powders or granules. The modulators may also be administered parenterally, either subcutaneously, intravenously, intramuscularly, intrasternally, transdermally or by infusion techniques. The modulators may also be administered as suppositories. A physician will be able to determine the required route of administration for each particular patient.

The formulation of a modulator for use in preventing or treating the conditions described above will depend upon factors such as the nature of the exact inhibitor, whether a pharmaceutical or veterinary use is intended, etc. An modulator may be formulated for simultaneous, separate or sequential use.

An modulator is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of a modulator is administered to a patient. The dose of a modulator may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

The invention potentially allows for the specific regulation of expression and/or activity of a particular isoform of NOS. Substances which have effects specific for one particular methylarginase isoform, for example a DDAHI or a DDAHII, may be administered non-specifically as they will only modulate the expression or activity of a particular methylarginase and thus the activity of one particular isoform of NOS.

Some substances may, however, have affect more than one isoform of methylarginase. Such modulators may have to be administered to specific sites, if they are required to regulate only one particular isoform of NOS. For example, if a condition requires the regulation of nNOS the modulator will have to be delivered to neurons. This may be achieved, for example, by delivery via a viral strain such as herpes simplex virus. Viral vectors comprising polynucleotides of the invention are described above. The viral vector delivery method may be used in the case of administration of, for example, polynucleotides of the invention.

The polynucleotides and vectors of the invention may be administered directly as a naked nucleic acid construct. Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™).

Typically, nucleic acid constructs are mixed with the transfection agent to produce a composition. Preferably the naked nucleic acid construct, viral vector comprising the polynucleotide or composition is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, or transdermal administration.

The pharmaceutical composition is administered in such a way that the polynucleotide of the invention, viral vector for gene therapy, can be incorporated into cells at an appropriate area. When the polynucleotide of the invention is delivered to cells by a viral vector, the amount of virus administered is in the range of from 10⁶ to 10¹⁰ pfu, preferably from 10⁷ to 10⁹ pfu, more preferably about 10⁸ pfu for adenoviral vectors. When injected, typically 1-2 ml of virus in a pharmaceutically acceptable suitable carrier or diluent is administered. When the polynucleotide of the invention is administered as a naked nucleic acid, the amount of nucleic acid administered is typically in the range of from 1 μg to 10 mg.

Where the polynucleotide giving rise to the product is under the control of an inducible promoter, it may only be necessary to induce gene expression for the duration of the treatment. Once the condition has been treated, the inducer is removed and expression of the polypeptide of the invention ceases. This will clearly have clinical advantages. Such a system may, for example, involve administering the antibiotic tetracycline, to activate gene expression via its effect on the tet repressor/VP16 fusion protein.

The use of tissue-specific promoters will be of assistance in the treatment of disease using the polypeptides, polynucleotide and vectors of the invention. It will be advantageous to be able express therapeutic genes in only the relevant affected cell types, especially where such genes are toxic when expressed in other cell types.

The routes of administration and dosages described above are intended only as a guide since a skilled physician will be able to determine readily the optimum route of administration and dosage for any particular patient and condition.

The invention is illustrated by the following Example:

### EXAMPLE

Materials and Methods

Unless otherwise indicated, the methods used are standard biochemistry and molecular biology techniques. Examples of suitable methodology textbooks include Sambrook et al., Molecular Cloning, A Laboratory Manual (1989) and Ausubel et al., Current Protocols in Molecular Biology (1995), John Wiley and Sons, Inc.

Database Searching and cDNA Cloning

The cDNA sequence of human DDAHI was obtained by a combination of database searching, specific RT-PCR and 5′/3′ RACE. The database of expressed sequence tags (dbEST) was searched with the cDNA sequence corresponding to the open reading frame of rat DDAHI (Kimoto, M., Sasakawa, T., Tsuji, H., Miyatake, S., Oka, T., Nio, N. and Ogawa, T., 1997, Biochim. Biophys. Acta 1337, 6-10) using the ‘blast’ programme. This search identified a single human cDNA sequence that comprised 161 bp of human DDAHI cDNA fused downstream of 160 bp of unknown sequence. Using this sequence, two human DDAHI specific oilogonucleotide primers HDDAHI.1 and HDDAHI.2 were designed. Human kidney polyA+RNA was reverse transcribed from an oligo dT primer, following which human DDAHI cDNA was PCR amplified in two PCR reactions incorporating either HDDAHI.1 and RDDAHI.1 or HDDAHI.2 and RDDAHI.2. In order to determine the sequence of the 5′ and 3′ ends of the human DDAHI open reading frame 5′ and 3′ RACE was performed. For 5′ RACE human kidney polyA mRNA was reverse transcribed using primer HDDAHI.3. Following reverse transcription, RNA was digested with RNAse H and cDNA purified using a HighPure DNA purification kit (Boehringer). Purified cDNA was polyA tailed by incubation with terminal transferase in the presence of dATP. Tailed cDNA was used directly in PCR reactions incorporating OligodTAnchor and HDDADI.4. For 3′ RACE human polyA+RNA was primed with OligodTAnchor and reverse transcribed prior to PCR with oligos HDDAHI.5 and Anchor. All PCR products were cloned into pCRTOPO2.1 (In Vitrogen) following the manufactures instructions. CDNA inserts were sequenced using a T7 sequences kit (Amersham) according to the manufacturers instructions.

The sequence of human DDAHII was obtained by data base searching. The database of translated EMBL open reading frames (trembl) was searched with the rat DDAHI peptide sequence. This search identified a hypothetical mouse open reading frame (accession number O08972) that has the capacity to encode a protein of 228 amino acids with 63% similarity to rat DDAHI. Interogation of dbEST with the nucleotide sequence encoding the hypothetical mouse protein identified numerous overlapping human EST's which contained an open reading frame of 858 bp with the potential to encode a 285 amino acid protein that is 52% identical to human DDAHI. The oligonucleotides used in these experiments are shown in Table 1.

TABLE 1
Oligonucleotides used.

| | | |
|---|---|---|
| Name | Sequence | Details |

| | | |
|---|---|---|
| HDDAHI.1 | GGT TGA CAT GAT GAA | Homologous to |
| | AGA AGC | nucleotides |
| | | 303-324 of human |
| | | DDAHI |
| HDDAHI.2 | CAG CAC CCC GTT GAT | Homologous to |
| | TTG TC | nucleotides |
| | | 454-435 of human |
| | | DDAHI |
| HDDAHI.3 | GCT TCT TTC ATC ATG | Homologous to |
| | TCA ACC | nucleotides |
| | | 324-303 of human |
| | | DDAHI |
| HDDAHI.4 | CCC AAC AAA GGG CAC | Homologous to |
| | GTC TTG | nucleotides |
| | | 682-703 of human |
| | | DDAHI |
| HDDAHII.1 | GAT CGA ATT CAG GAT | Homologous to |
| | GGG GAC GCC GGG G | nucleotides |
| | | −2-15 of human |
| | | DDAHII encoding an |
| | | upstream Eco RI site |
| HDDAHII.2 | GAC TTC TAG AGC TGT | Homologous to |
| | GGG GGC GTG TG | nucleotides |
| | | 858-840 of human |
| | | DDAHII encoding a |
| | | downstream XbaI site |
| HDDAHII.3 | CTC AGC TCC CTC TGC | Homologous to |
| | TTG GTG | nucleotides |
| | | 813-834 of human |
| | | DDAHII |
| HDDAHII.4 | GAG GGA GGA TTC ACC | Homologous to |
| | CAG TGG | nucleotides |
| | | 1003-1024 of human |
| | | DDAHII |
| RDDAHI.1 | TCC GCG GGA TCC ATG | Homologous to |
| | GCC GGC CTC | nucleotides |
| | | −12-12 of rat |
| | | DDAHI |
| RDDAHI.2 | CGC TCG GTC TAG ATC | Homologous to |
| | AAG AGT CTG TCT T | nucleotides |
| | | 872-844 of rat |
| | | DDAHI |
| HNNOS.1 | CTG CTG ATG TCC TCA | Homologous to |
| | AAG CCA TCC | nucleotides |
| | | 4079-4102 of human |
| | | nNOS |
| HNNOS.2 | TCT GTC CCG CGC TTA | Homologous to |
| | CAA ACT TGC | nucleotides |
| | | 4353-4330 of human |
| | | nNOS |
| HENOS.1 | CAA CCA ACG TCC TGC | Homologous to |
| | AGA CCG TGC | nucleotides |
| | | 3379-3402 of human |
| | | eNOS |
| HENOS.2 | GGC GGA CCT GAG TCG | Homologous to |
| | GGC AGC CGC | nucleotides |
| | | 3690-3667 of human |
| | | eNOS |
| Oligo d(T) | GAC CAC GCG TAT CGA | 5′/3′ RACE oligo |
| Anchor | TGT CGA CTT TTT TTT | d(T) anchor primer |
| | TTT TTT TTV | |
| Anchor | GAC CAC GCG TAT CGA | 5′/3′ RACE anchor |
| | TGT CGA C | primer |

Recombinant Expression

The entire human DDAHII open reading frame was PCR amplified from oligo dT primed human kidney cDNA using oligos HDDAHII.1 and HDDAHII.2. Oligo HDDAH II.1 is complementary to base pairs 2-15 of the human DDAHII cDNA and contains an EcoRI site in frame with the EcoTI site of pPROX.HTa (Life Technologies). HDDAHI.2 is complementary to base pairs 858-840 of the human DDAHII cDNA and contains an artificial XbaI site. PCR produced a single product of ˜850 bp which was digested with EcoRI and Xbai, ligated into EcoRI and XbaI digested pRPROX.HTa and transformed into compotent E.coli DH5α. A positive clone (pPDDAHII) containing an insert of 858 bp was identified and the insert sequenced on both stands. For expression of recombinant human DDAHII, E.coli were grown in liquid culture at 25 ° C. to an OD₆₀₀ of 0.5-0.6. Expression was then induced by the addition of IPTG to a final concentration of 1 mM and incubation continued for a further two hours. Following induction, cells were collected by centrifugation, weighed and resuspended in ice cold assay buffer (100 mM Na₂HPO₄ pH 6.5) at 1 g cells/ml. Cells were disrupted by sonication (6×10 secs, with 10 sec. intervals) and centriguged at 50,000 g to separate soluble material from insoluble cell debris.

DDAH Assay

Aliquots of E.coli lysates were incubated at 37° C. for 60 min. with 250 ml of 100 mM Na₂HPO₄ pH 6.5 containing 0.02 μCi [¹⁴C]L-NMMA as described previously (MacAllister et al., 1996, Br. J. Pharmacol. 115, 1001-1004). Following incubation samples were prepared for determination of [¹⁴C] citrulline production by scintillation counting. Reactions were vortexed with 1 ml of 50% (w/v) dowex 50X8-400, centrifuged at 10,000 g for 5 min and then 500 μl of the supernatant was mixed with 5 ml of liquid scintillation fluid and the [¹⁴C] content determined.

DDAH Assay

Samples were assayed by incubating 100 μl of cells lysates with an equal volume of assay mix (100 mM Na₂HPO₄ pH 6.5, containing 0.02 μCi [¹⁴C] L-NMMA and 100 μM cold L-NMMA) at 37° C. for 60 min, as previously reported. The samples were then prepared for scintillation counting to measure the production of [¹⁴C] citrulline by adding 400 μl of 50% (w/v) Dowex 50X-400 to the reactions, vortexing and centrifugation at 13000 g for 2 min. The [¹⁴C] content of 100 μl of supernatant in 1 ml scintillation fluid was then determined.

Northern Blot Analysis

The tissue distribution of human DDAHI, DDAHII, endothelial NOS and neuronal NOS mRNA was determined by hybridization of ³²P-labelled cDNA probes to commercially available northern blot (Clontech, human multiple tissue northern blot). Probes were produced by PCR amplification of oligo dT-primed human kidney polyA+mRNA using oligonucleotide primer pairs HDDAHI 4 and 5, HDDAHII 3 and 4, HENOS i and 2 and HNNOS 1 and 2. Following PCR reaction products were resolved on 2% agarose gels, isolated from the gel and labelled using a random primed labelling kit (Boehringer) according to the manufacturers instructions. Labelled probes were purified on Nick columns (Pharmacia) and hybridized to filters according to the manufacturers instructions.

Database Searching for Bacterial Sequences

The amino acid sequence of human DDAHI was added to search the expressed sequence tag database (dbEST). Open reading frames which showed significant similarity to this sequence were identified in S.coelicolor (46.5% over 163 amino acids), P.auruginosa (44.3% over 226 amino acids) and M.tuberculosis (37.5% over 24 amino acids). The cDNA sequences encoding these putative bacterial DDAHs were then used to design the primers ScDDAHI and ScDDAH2, PaDDAHs were then used to design the primers ScDDAHi and ScDDAH2, PADDAH1 and PaDDAH4, and TbDDAH1 and TbDDAH4. These oligonucleotides PaDEIM2 and PaDEIM3 were designed from the cDNA sequence of P.aeruginosa arginine deiminase to amplifying the coding region. Primers TbDDAH1 and TbDDAH4 were designed to amplify an open reading frame from cosmid Y3G12 which was identified through the database search using hDDAH1. The oligonucleotides used in these experiments are shown in Table 2.

TABLE 2
Oligonucleotides Used.

| | | |
|---|---|---|
| Name | Sequence | Details |

| | | |
|---|---|---|
| ScDDAH 1 | GATCGAATTGTGCCCAGCAA | Homologous to −9 to |
| | GAAG GCCTG | 20 encoding an |
| | | upstream Eco RI site |
| ScDDAH 2 | GATCTCTAGATCAGTCGTAC | Homologous to 732 to |
| | AGCTC GCGC | 751 encoding a |
| | | downstream XbaI site |
| PaDDAH 1 | GAATTCATGTTCAAGCACAT | Homologous to 1 to |
| | CATCG | 19 encoding an |
| | | upstream Eco RI site |
| PaDDAH 4 | AAGCTTCGCCGCGGCATGGT | Homologous to 782 to |
| | TC | 768 encoding a |
| | | downstream Hind III |
| | | site |
| TbDDAH 1 | GAATTCCGCAATGTATCAAT | Homologous to −12 to |
| | G G | 4 encoding an |
| | | upstream Eco RI site |
| TbDDAH 2 | AAGTTCCCACGCACCCTCAG | Homologous to 1024 to |
| | | 1011 encoding a |
| | | downstream Hind III |
| | | site |
| PaDEIM 2 | GAATTCAGCACGGAAAAACC | Homologous to 3 to |
| | AAAC | 22 encoding an |
| | | upstream Eco RI site |
| PaDEIM 3 | AAGCTTGTAGTCGATCGGGT | Homologous to 1257 to |
| | CGC | 1239 encoding a |
| | | downstream Hind III |
| | | site |

Polymerase Chain Reaction and cDNA Cloning

Amplification of S.coelicolar DDAH from cosmid 4C6 was carried out by PCR using the oligonucleotides ScDDAH1 and ScDDAH2. PCR was carried out on P.aeruginosa genomic DNA using the primers PADDAG1 and PADDAG4 to amplify the putative DDAH. The P.aeruginosa arginine deiminase was also amplified using the oligonucleotides PaDEIM2 and PaDEIM3. The oligonucleotides TbDDAH1 and TbDDAH4 were used in PCR to amplifying the M.tuberculosis DDAH from cosmid Y3G12 DNA.

All PCR products were cloned into pCRTOPO2.1 (In Vitrogen) following the manufacturer's instructions.

Expression of Recombinant Proteins

The inserts containing the open reading frames of the bacterial DDAHs were excised from the vector using Eco RI and Hind III, gel purified, ligated into Eco RI and Hind III digested pProEX.HT and transformed into competent E.coli DH5α. The arginine deiminase was treated as above but was cloned into EcoRI and Hind III digested pBAD B (In Vitrogen).

For expression of the recombinant proteins, a positive clone was picked and grown in liquid media supplemented with 100 μg/ml ampicillin. E.coli were grown at 25° C. to an OD₆₀₀ of 0.5-0.6 for the bacterial DDAHs in pProEX.HT, and at 37° for the arginine deiminase in pBAD B. Induction of expression of the bacterial DDAHs was carried out by addition of IPTG to a final concentration of 1 mM and a further incubation of 2 hours at 25° C. Expression of the arginine deiminase was induced by adding arabinose to a final concentration of 0.02% (w/v) and incubating fora further 4 hours at 37° C.

After induction, cells were harvested by centrifugation and resuspended to a concentration of 250 mg/ml in assay buffer (100 mM Na₂HPO₄, pH6.5). Cells were disrupted by sonication (6×10 secs.) And centrifuged at 18,000 g to remove particulate material.

SDS-Polyacrylamide Gel Electrophoresis and Western Blot Analysis

SDS-PAGE was performed in Tris/glycine buffer, pH8.3, on 12% (w/v) separating gel with a 3.5% (w/v) stacking gel. Proteins were transferred onto an Immobilon-P membrane (Millipore) at 200 A for 30 minutes. Membranes were then blocked in 5% (w/v) milk in phosphate buffered saline with 0.1 Tween 20 (PBST) for 2 hours. The blot was probed with a polyHistidine antibody (Sigma) at a dilution of 1:3000 followed by anti-mouse Ig antibody coupled to horseradish peroxide (Amersham) at a dilution of 1:5000 then developed using an ECL chemiluminescence kit (Amersham).

Results

Cloning of Human DDAHI and DDAHII

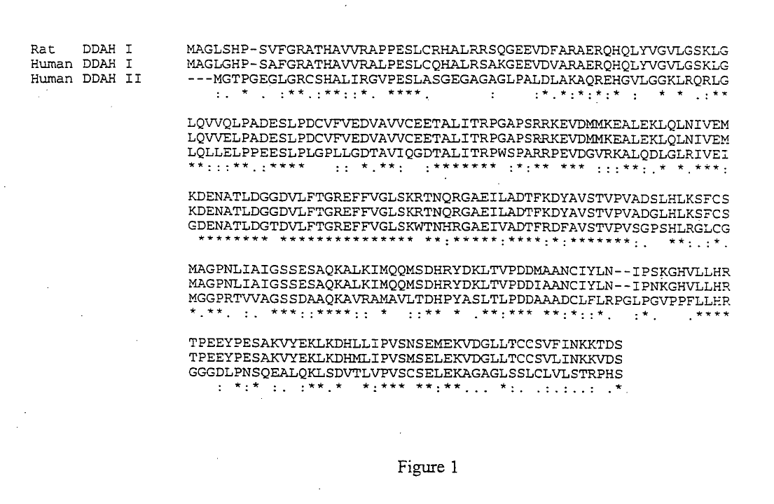

Using a combination of RT-PCR and RACE, a cDNA encoding the entire open reading frame of human DDAHI was assembled. The 858 bp open reading frame is 90% homologous to rat DDAHI ORF (data not shown) and encodes a polypeptide of 285 amino acids that is 95% identical to the rat protein (FIG. 1). A search of the ‘trembl’ data base using the rat DDAHI amino acid sequence identified a mouse open reading frame encoding a protein with 63% homology over 228 amino acids to rat DDAH. Further data base searching identified a human cDNA of 2000 bp containing an open reading frame of 858 bp with the potential to encode a protein of 285 amino acids (subsequently referred to as DDAHII). This open reading frame was 63% homologous to human DDAHI at the nucleotide level (data not shown) and the predicted protein is 62% similar to human DDAHI at the amino acid level (FIG. 1). Like DDAHI, DDAHII appears to be highly conserved across mammalian species with 98& homology between murine and human DDAHII amino acid sequences (data not shown).

Recombinant Expression of Human DDAHII

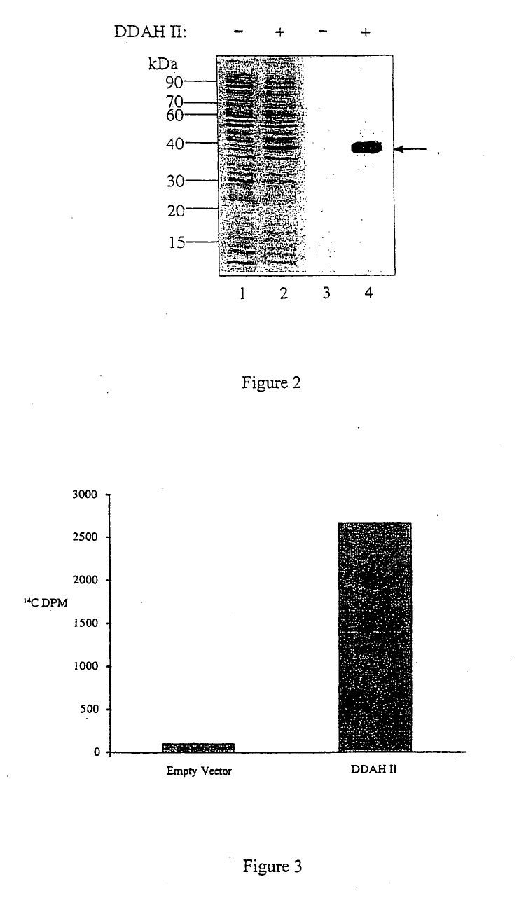

An N-terminally 6X His-tagged body of DDAHII was expressed in E. coli under the control of an IPTG induceable promoter. Following induction, a band of ˜40 kDa (−35 kDa human DDAHII+4 kDa 6X His-tag and linker) was apparent in the soluble fraction of cell lysates (FIG. 2). The induced protein of ˜40 kDa is specifically recognised by an anti-His6 antibody confirming its identity as recombinant human DDAHII (FIG. 2). In order to establish whether DDAHII is a functional homologue of DDAHI we assayed bacterial cell lysates for DDAH activity. Lysates of cells transfected with empty vector did not metabolise [¹⁴C] L-NMMA. In contrast, lysates of cells expression recombinant DDAHII did metabolise [¹⁴C] L-NMMA (FIG. 3). This action was inhibited by the DDAH inhibitor S-2-amino-4(3-methylguanidino)butanoic acid (4124W) [ref] and by competition with a molar excess of cold L-NMMA, ADMA or citrulline. Enzyme activity was unaffected in the presence of a molar excess of cold SDMA.

Tissue Distribution of Human DDAH and NOS

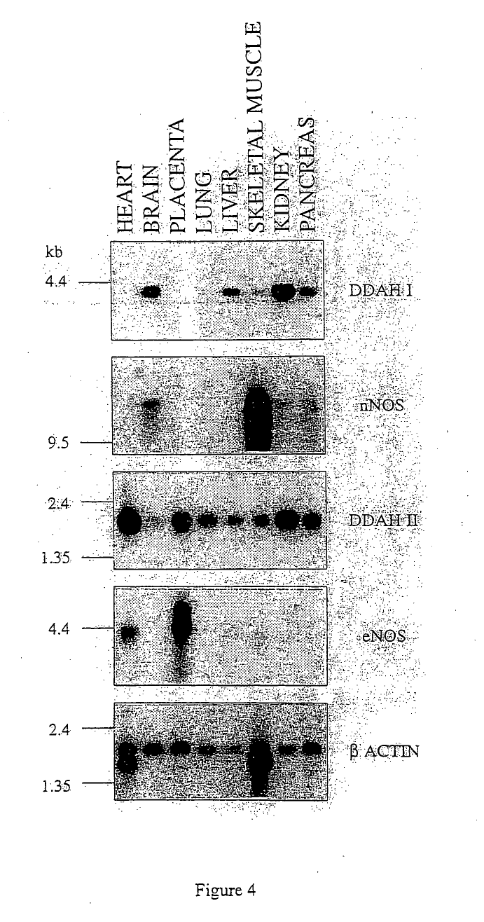

To determine the tissue distribution of DDAHI and DDAHII messenger RNA and to explore any correlation between DDAH and NOS isoform expression we probed a commercially available human multiple tissue northern blots with labelled cDNA probed specific for each isoform (FIG. 4). A DDAHI cDNA probe hybridized to a single band of ˜4.4 Kb that was highly expressed in kidney, brain, pancreas and liver. Lower level expression was also clearly apparent in skeletal muscle whilst signals from the heart placenta and lung were barely detectably. In contrast, a cDNA probe for DDAHII hybridized to a single band of ˜2 Kb that was most highly expressed by heart, kidney and placenta. In the case of DDAHII, lower level expression in the brain was barely detectable. A probe specific for nNOS revealed high level expression in skeletal muscle and brain, lower levels in kidney and pancreas with no detectable expression in heart, placenta, lung and liver. Endothelial NOS was highly expressed in placenta and heart with lower levels apparent in skeletal muscle, liver, kidney, pancreas and lung, whilst expression in brain was undetectable. The level of β-actin message in each lane is shown as an indication of mRNA loading.

To extend this analysis of DDAH isoform distribution we have examined the level of DDAH I and II mRNA in 43 adult and 7 fetal tissues by RNA dot blot analysis using isoform specific probes. This analysis indicated that in the brain DDAHI is expressed at high levels whilst DDAHII is expressed at relatively low levels, with both isoforms being expressed at high levels in the spinal cord. However, in highly vascularised tissues such as the heart, aorta, placenta and lung DDAHII expression clearly predominates over DDAHI. In immune tissues (spleen, thymus, peripheral leukocyte, lymph node and bone marrow) DDAHII expression is clearly apparent whilst DDAHI expression is barely detectable. This distinct pattern of DDAH expression is consistent with the suggestion above that DDAHI predominates in tissues which express high levels of the neuronal isoform of NOS and DDAHII expression is most marked in tissues that also express the endothelial form of NOS.

The Chromosomal Location of Human DDAHI and II

In order to determine the chromosomal location of the human DDAHI and DDAHII genes we employed radiation hybrid mapping and fluorescent in situ hybridisation (FISH) techniques. For radiation hybrid mapping we screened the Genebridge 4 Radiation Hybrid DNA Panel (Originators Peter Goodfellow and Jean Weissenbach, obtained from the UK HGMP Resource Centre) using oligonucleotide pairs to amplify nucleotides 47 to 273 of the DDAHI cDNA and nucleotides −16 to −163 of the DDAHII cDNA. The results were submitted to the HGMP RhyME mapper which mapped DDAHI to chromosome 1 (LOD=2.7) and DDAHII to chromosome 6p21-23 (LOD>3). For FISH mapping, a human DDAHI genomic clone (clone no. 2218H15, accession number AQ145822) was identified by a blast search of the GSS database using the human DDAHI cDNA sequence. A genomic clone (clone no. 84h3) containing the human DDAHII gene was isolated by PCR screening of a human genomic PAC library (RPCI1, onstructed by Pieter de Jong and obtained from the UK HGMP resource Centre). Clones 2218H15 and 84h3 were used as probes in FISH which localised DDAHI to chromosome 1p22 and DDAHII to chromosome 6p21.3.

Identification of DDAH-Related Proteins

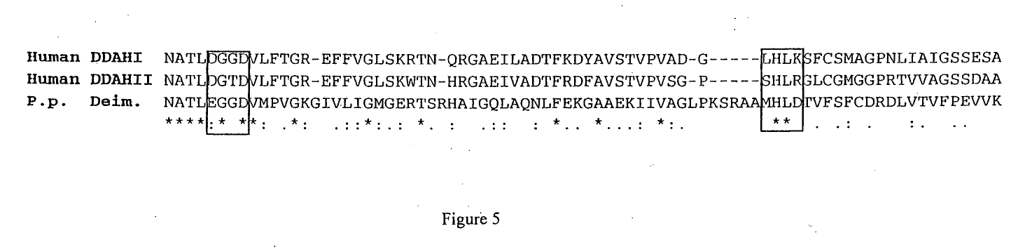

In order to identify proteins with significant primary sequence homology to DDAHI/II we performed a search of the swissprot data with both the human DDAHI and DDAHI protein sequences. This search revealed significant homology between both DDAH sequences and the sequences of arginine deiminase enzymes from several microbial species. The highest degree of homology was found with the sequence of arginine deiminase from Pseudomonas putida (Accession no. p41142) (FIG. 5). The homology was strongest within a 69 amino acid domain (residues 123 to 191 of DDAHI) where the identify rises to 22% and the similarity to 70%. In this domain, DDAHI and DDAHII are 80% identical. Comparison of the sequences of human DDAHI and DDAHII with other arginine-utilizing or arginine-producing enzymes, such as peptidyl-arginine deiminase, arginase, argininosuccinate lysase, arginine decarboxylase and nitric oxide synthase revealed no significant amino acid homology.

Cloning of Streptomyces and Pseudomonas DDAH

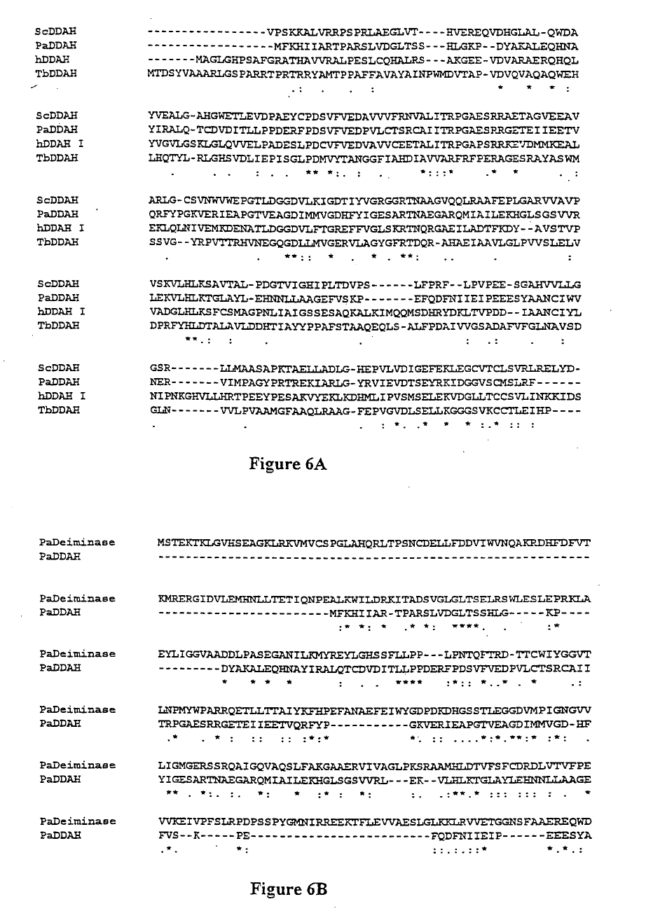

A ClustalW alignment of the DDAHs from S. coelicolor, P. aeruginosa, M. tuberculosis and human DDAH I amino acid sequences is shown in FIG. 6A. Alignments of P. aeruginosa DDAH and arginine deiminase are also shown in FIG. 6B.

Oligonucleotides ScDDAH 1 and ScDDAH 2 were designed from the open reading frame of a putative S. coelicolor DDAH identified through database screening. These primers gave a PCR product of approximately 850 bp. The primers PaDDAH 1 and PADDAH 4 amplified a product of approximately 780 bp from P. aeruginosa genomic DNA and TbDDAH 1 and TbDDAH 4 gave a PCR product of approximately 1150 bp from the cosmid Y3G12.

Expression of Recombinant Bacterial DDAHs

Expression of N-terminally 6X His-tagged forms of S. coelicolor, M. tuberculosis and P. aeruginosa DDAH was carried out in E. coli under the control of an IPTG inducible promoter. Following induction, a band of ˜36 kDa was observed in S. coelicolor (˜32 kDa S. coelicolor DDAH+˜4 kDa 6X His-tag) cell lysates and of 33 kDa in P. aeruginosa (29 kDa P. aeruginosa DDAH+˜4 kDa 6X His-tag) cell lysates. A polyHistidine antibody specifically recognized these bands providing confirmation of the identity of these proteins as recombinant S. coelicolor and P. aeruginosa DDAH respectively.

Activity of Recombinant Bacterial DDAH Proteins

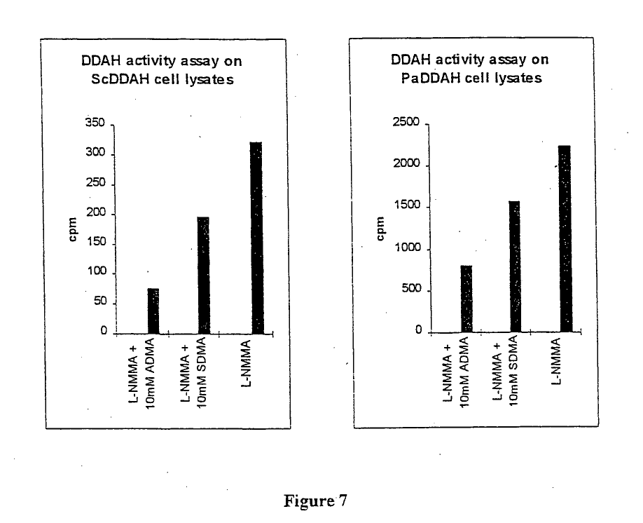

The bacterial DDAH cell lysates were assayed for DDAH activity to determine whether they were functional homologues of human DDAH I. These were found to metabolize [₁₄C] L-NMMA, as shown in FIG. 7. Empty vector was also transfected into cells and the lysates from these were found not to metabolize [₁₄C] L-NMMA. P. aeruginosa DDAH showed higher activity compared to that of S. coelicolor DDAH.

ADMA and SDMA were both found to compete with L-NMMA as substrates for the bacterial DDAHs (FIG. 7) with ADMA showing a greater effect than SDMA on the metabolism of L-NMMA. Similar results have been obtained for DDAH from M.tuberculosis.

1-45. (canceled)

46. An isolated polynucleotide which:
(a) encodes a polypeptide that has the properties of a methylarginase, which polynucleotide is selected from the group consisting of:
(1) the coding sequence of SEQ ID NO:3;
(2) a fragment of a sequence defined in (1);
(3) a sequence which hybridises selectively to the complement of a sequence defined in (1) or (2); and
(4) a sequence that is degenerate as a result of the genetic code with respect to a sequence defined in (1), (2) or (3); or
(b) is a sequence complementary to a polynucleotide defined in (a).

47. An isolated polynucleotide according to claim 46 which is a DNA sequence.

48. An isolated polynucleotide according to claim 46 which encodes the amino acid sequence of SEQ ID NO:4 or is a sequence complementary thereto.

49. A vector incorporating a polynucleotide which:
(a) encodes a polypeptide that has the properties of a methylarginase, which polynucleotide is selected from the group consisting of:
(1) the coding sequence of SEQ ID NO:3;
(2) a fragment of a sequence defined in (1);
(3) a sequence which hybridises selectively to the complement of a sequence defined in (1) or (2); and
(4) a sequence that is degenerate as a result of the genetic code with respect to a sequence defined in (1), (2) or (3); or
(b) is a sequence complementary to a polynucleotide defined in (a).

50. A vector according to claim 49 which is an expression vector.

51. An isolated cell harbouring a polynucleotide which:
(a) encodes a polypeptide that has the properties of a methylarginase, which polynucleotide is selected from the group consisting of:
(1) the coding sequence of SEQ ID NO:3;
(2) a fragment of a sequence defined in (1);
(3) a sequence which hybridises selectively to the complement of a sequence defined in (1) or (2); and
(4) a sequence that is degenerate as a result of the genetic code with respect to a sequence defined in (1), (2) or (3); or
(b) is a sequence complementary to a polynucleotide defined in (a), or a vector incorporating a said polynucleotide.

52. A process for the preparation of a polypeptide which has methylarginase activity, which process comprises the steps of cultivating a host cell harbouring an expression vector according to claim 50 under conditions to provide for expression of the said polypeptide, and recovering the expressed polypeptide.

53. A method for identifying a modulator of methylarginase activity and/or expression, comprising the steps of:
(i) contacting a test substance and a cell harbouring a polynucleotide which:
(a) encodes a polypeptide that has the properties of a methylarginase, which polynucleotide is selected from the group consisting of:
(1) the coding sequence of SEQ ID NO:3;
(2) a fragment of a sequence defined in (1);
(3) a sequence which hybridises selectively to the complement of a sequence defined in (1) or (2); and
(4) a sequence that is degenerate as a result of the genetic code with respect to a sequence defined in (1), (2) or (3); or
(b) is a sequence complementary to a polynucleotide defined in (a), under conditions that in the absence of the test substance would permit expression and activity of the polypeptide encoded by the said polynucleotide; and
(ii) determining thereby whether the said substance modulates the activity and/or expression of methylarginase.

54. A vector according to claim 49, wherein the polynucleotide encodes the amino acid sequence of SEQ ID NO:4.

55. An expression vector according to claim 50, wherein the polynucleotide encodes the amino acid sequence of SEQ ID NO:4.

56. An isolated cell according to claim 51, wherein the polynucleotide encodes the amino acid sequence of SEQ ID NO:4.
